(19) United States
(12) Patent Application Publication
(10) Pub. No.: US 2012/0083542 A1
(43) Pub. Date: Apr. 5, 2012

(54) METHOD FOR TREATING WASTES

(75) Inventor: Thomas Müller, Dresden (DE)

(73) Assignee: PATRICK VON HERTZBERG, Potsdam (DE)

(21) Appl. No.: 13/139,053

(22) PCT Filed: Mar. 18, 2009
(86) PCT No.: PCT/EP2009/001991

§ 371 Date: Nov. 23, 2011

(30) Foreign Application Priority Data
Dec. 11, 2008 (DE) 10 2008 055 508.8

(51) Int. Cl. C08J 11/04; C08H 7/00; C07C 51/41; C02F 1/68; C07K 2/00; C07C 43/29; B01J 19/00; C08B 1/00; C07C 29/00

(52) U.S. Cl. 521/47; 521/48; 521/46.5; 521/49; 521/49.8; 536/56; 530/500; 554/156; 568/852; 530/402; 568/639; 422/184.1; 422/187; 210/766

(57) ABSTRACT

The present invention relates to a process for the treatment of waste in a reactor (3) comprising the steps of combining the waste to be processed and an aqueous, alkaline solution to form a reaction mixture, heating the reaction mixture to a temperature range between 140° C. to 250° C. and a pressure between 3 bar and 12 bar to produce at least one aqueous, organic phase, a gas phase and optionally a solid phase, removing the gas phase from the reactor (3), separating the organic and aqueous components of the gas phase, in particular by means of a rectification column and feeding the aqueous components of the gas phase back into the reactor.

The invention relates to a process for treatment of waste.

Means of substance utilization known from the prior art have been limited. Processes are known attempting to use hydrodynamic differences to separate components (e.g. classification), or processes based on instrumental analytical methods (e.g. spectroscopic), or processes based on density differences (float-sink plants) or processes based on differences in solubility of certain plastics in—mostly—organic solvents.

All these alternatives have in common that they either result in unsatisfactory separation and/or they allow only for material utilization, if allowing for substance utilization at all.

Furthermore, processes are known that use thermo-chemical transformations, e.g. gasification, pyrolysis, reforming, etc., to generate cleavage products and/or transformation products that may be used for energy and/or as feedstocks (e.g., synthesis gas, monomers).

Also, processes are known that may result in the formation of usable products by hydrogenation under forcing conditions (Bergius principle).

Most established waste treatment processes are based on material utilization by mechanical/physical separation of waste.

Utilizable materials reclaimed in such manner, such as films, PVC, etc., are sold as relatively pure fractions or as processed secondary granules. A portion of this material is processed into new products, which usually represent a down-cycling, with just a small fraction of it having real market opportunities.

Residual fractions that remain after sorting out, are processed mostly as alternative fuels and provide electricity and heat by subsequent utilization of the chemical energy contained in the waste by means of combustion.

Mostly, however, waste disposal is the focus rather than utilization for the generation of energy.

Ultimately, manufacturing costs associated with waste plastics are always lost in combustion, since they are usually significantly higher than the calorific value.

DE 197 50 327 C1 discloses a process for producing synthesis gas from renewable cellulose-containing feedstock or waste materials that is due to its consistency particularly suited for subsequent combustion in internal combustion engines. Contaminated cellulose-containing materials, such as waste wood, are utilizable in this process. A recycling of mixed waste is only conditionally possible.

Based on the foregoing, it was the object of the present invention to provide, independent of the proportion of each component, a high degree of utilization of the substances contained in the waste materials and/or of the energy releasable therefrom. In particular, the invention is allowing recovery substances from the waste. Here, the focus is on, inter alia, shredder residue of car- and mixed waste recycling, the material utilization of which is currently difficult, and on other small-particle bulk waste with a high, often elusive problem fraction (PVC, flame retardant, etc.).

This task is solved by the process having the features of claim 1. Further dependent claims illustrate advantageous embodiments.

Within the scope of the present application, waste is understood to mean the totality of all household waste, production waste, industrial waste or production residues the owner wants or has to dispose of such as:
solid and liquid biomasses or products produced thereof,
plastics (polymers),
elastomers,
inorganic materials such as minerals, metals and compounds thereof,
solid or liquid pollutants and/or hazardous substances, such as heavy metals and compounds thereof,
halogen-substituted organic compounds such as flame retardants,
water (moisture).

In the process according to the invention, the waste is treated initially with an alkaline solution under pressure and at a temperature between 140° C. and 250° C. of the alkaline solution and at the same time, or subsequently, the inorganic constituents are separated. Furthermore, the organic components of the resulting dragging steam are separated from the water and subsequently processed further into fuel gas or synthesis gas by rectification, extraction or sorption and/or conversion by means of thermal evaporation. The first step is based on treating organic waste components by means of solvolysis, such that subsequently they are present in at least one liquid phase. Hereinafter, the chemical reactions that individual components of the waste are undergoing are described by way of example.

Solvolysis is understood to mean any chemical reaction taking place between the waste and the alkaline solution.

According to the process according to the invention, the waste is combined in a reactor with an aqueous, alkaline solution and an essentially water-insoluble organic auxiliary to form a reaction mixture. The reaction mixture is heated to a temperature range between 140° C. to 250° C. and pressured between 3 bar and 12 bar to produce at least, an aqueous, an organic phase, a gas phase and optionally a solid phase. The gas phase is removed from the reactor and the organic and aqueous components of the gas phase are separated. Subsequently, the aqueous components of the gas phase are fed back into the reactor.

Advantageous alkaline solutions are those containing inorganic carbonates. Particularly advantageous is potassium carbonate solution.

Preferably, the alkaline carbonate solutions are saturated having a density between 1.5 to 1.6 g/cm³.

Preferably, the organic auxiliaries are lubricating oils, motor oils, aliphatic hydrocarbons or the like. Preferably, the organic auxiliaries are inert to the alkaline solution.

Organics, i.e., the organic components which are dissolved mainly in the organic auxiliary and which do not have a sufficient vapor pressure to be discharged with the dragging steam, are predominantly nonpolar and float in the alkaline solution, since their density is smaller than the density of the alkaline solution.

Preferably, the floating phase is discharged in a batch process with stirrer tank reactor, and in the case of the semi-batch reactor with continuous substance supply when the entire solution is pumped off or pumped into another container. Here, the solution may be pumped into separate containers by means of a 3-way valve in dependence of its conductivity (oil phase has low conductivity, aqueous phase has high conductivity).

Water usually enters the process in the form of moisture. Depending on the composition of the starting material and the target products water is an important parameter Processes take place consuming water, e.g. hydrolyses, or eliminating water from. Very dry starting material usually requires the addition of a small amount of water.

In liquid one-component systems, the vapor pressure of the liquid is only a function of temperature. This saturation pressure is described by the Clausius-Clapeyron equation. In multi-component systems, the relations are more complex. If the components are immiscible, usually the vapor pressures of the individual components are approximately additive (Dalton's law). For the ionic components, which are formed in the present case, by the ions of the dissolved potassium carbonate and the organic components present in the solution in ionic form, other laws apply. Here, components having lower vapor pressure than water have a vapor pressure-lowering effect. These colligative properties are generally described by Raoult's law, but have only limited applicability at such high concentrations as in the present case.

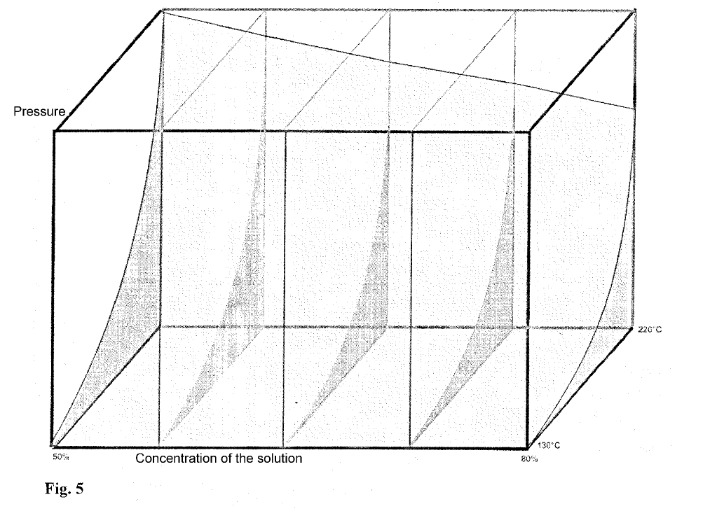

In a particularly preferred embodiment, the salt concentration has an impact on the operation of the process, as shown in FIG. 5.

Thus, the solution concentration and temperature are crucial parameters for the vapor pressure, i.e., the system pressure. Of course, this applies only on the condition that gaseous reaction products, such as CO₂ and NH₃ are removed from the system.

From the perspective of control, it is preferred to control temperature and pressure to obtain the corresponding concentration, i.e., excess water leaves the system. Only when water-consuming processes, e.g. hydrolyses, predominate, an addition of water is necessary. By comparison with the p/T curve, this may be determined by ICA.

In addition to impacting the equilibrium pressure in the system, the reaction temperature mainly impacts the following parameters:
the kinetics of the solvolysis processes,
the secondary decomposition reactions,
the viscosity of the raw materials and
the partial pressure of the organic components in the dragging steam.

Preferably, the reaction temperature is 140 to 250° C.

Preferably, the reaction times are in the range of 0.5 to 30 hours (batch mode) and the equilibrium pressures are preferably at 3 to 12 bar, particularly 4 to 10 bar.

The solvolysis can be performed as a batch processes or quasi-continuous semi-batch processes in a stirrer tank or loop reactor or as a combination of both. Both options have advantages. The selection is made depending on the expected input. Is the precursor very fragmented and ductile, the continuous variant offers big advantages in terms of space/time yield. For small plants with remote monitoring more likely a batch operation will prove to be advantageous.

Preferably, the waste material is stirred during solvolysis. This may be carried out by means of a mechanical stirrer, e.g. anchor stirrer, or by moving the liquid in the circulating reactor. Here also the selection is made depending on the expected input.

The discharge of the solid phase can also be carried out batch-wise or continuously. A batch-wise discharge is appropriate if there is a risk of long wires/cables being present in the waste material.

Preferably, after being discharged, the organic ingredients are washed with processed, alkaline solution and/or water. Washing the inorganic components is a preferred process step, as digestion solution is frequently found in the interspace of the inorganic constituents either bound by adhesion or fixed by adsorption. Subsequently, the wash water can be fed back into the digestion solution.

A rectification column is directly connected to the reactor, wherein, preferably, the sump of the column is spatially separated from the reactor. After the column (i.e., in series), preferably, a pressure regulator is arranged that maintains the system pressure constant at the equilibrium pressure. In this manner, gases released during solvolysis that are formed only in very small quantities, are discharged from the system.

It is further preferred that organic compounds are discharged from the system by extraction processes, preferably using oils. Optionally, it is also possible here to increase the yield of the extraction or distillation by means of hydrolysis or electrolysis.

The task of the rectification column is to separate the organic components which are obtained according to their vapor pressure (partial pressure) and whose vapor pressure is lower than that of water, from the dragging steam above the sump, and to remove them from the system. By doing so, the thermodynamic system is permanently disturbed and organics are “replenishing” the steam. In this manner, the organic components are discharged in a potassium-free form via the sump of the column.

Preferably, water vapor loaded with the organic constituents is fed from the bottom via the sump into a column, passes through the packing to the top and the organic constituents are depleted in the steam by alternating condensation and evaporation processes. Next, the steam leaves the column at the top end. On the other hand, the separated organic ingredients flow into the sump.

Preferably, by the top condensate as much condensation heat must be removed by the condensation of water, as the condensing organic components are able to transfer to this water in the form of heat of vaporization.

Preferably, the organic components are removed from the solvolysis reactor by means of a column and/or by means of extraction.

The goal is to obtain potassium-free organic compounds with low viscosity, which then can be separated due to substance-specific properties, such as vapor pressure, polarity, dielectric properties, density, etc.

A separation by means of a column requires a sufficient vapor pressure, while extraction requires a largely non-polar behavior of the organic components.

A too low vapor pressure particularly results from too large molecules and/or too strong intra- and intermolecular interactions. Strong binding forces prevent the substance from entering into in the vapor phase. These binding forces (which are not true chemical bonds) are particularly strong in very polar substances, i.e., functional groups that contain heteroatoms. But not only the presence of hetero atoms alone, but also the corresponding binding modes, that is the nature of the chemical functionality, determines how strong the binding forces are. Strong binding forces cause a lowering of the vapor pressure, particularly, when the compounds are converted into their salt form (e.g., carboxylates, alkoxides, thiolates, etc.) i.e. by deprotonation.

In the case of a too large molecule size, substances with sufficiently low polarity are amenable to extraction, so that in this case overall only the decrease in the polarity must be achieved. A low polarity is a prerequisite for extractability.

Inter alia, there are two ways to decrease the polarity of the substance which are described hereinafter in more detail. The goal of both methods is to achieve a quantitative separation of the organic matter, so a thermal regeneration of the salt solution is not necessary.

By partial hydrogenation functional groups can be converted into a different “oxidation state”, which is less polar, e.g. from carboxyl to hydroxyl. Low acidities (KA value) obtained this way result in a higher proportion of a substance being present in a protonated form which has a vapor pressure.

This increases the partial pressure of the organic component in the vapor space, which makes the same amenable to rectification.

Furthermore, complete hydrogenation may also take place, eliminating the functionality in its entirety.

Preferably, Raney nickel as a suitable catalyst for the hydrogenation under the conditions present wherein it may also be activated in vitro, if applicable. It is particularly active in the alkali solution. Resulting water, H₂S NH₃, etc., are discharged by the pressure control and treated separately.

Particularly the very polar carboxyl groups that are highly prone to salt formation can be removed in a very elegant way. By a one-electron oxidation a radical decomposition is initiated electrochemically, which leads to decarboxylation and addition (in a special case to dimerization) of the residues. This mechanism is known as Kolbe electrolysis or Kolbe synthesis.

The extraction can be performed continuously in a circulation process or batchwise. In some cases, e.g. if the mixture contains large quantities of bitumen the use of additional extractants may be waived because pumpable viscosities have already been achieved. Also, when using certain feedstocks that are free from, for example, polyolefins, the use of extractants may be waived also.

Extractants should be chosen such that they are either separated easily from the extracted substance/mixture of substances, e.g., by means of vacuum distillation, or are further processed together with the extracted substance, e.g. by hydrocracking, so that again sufficient extractant is available. Also, inexpensive extractant, such as oil, may be used.

Materials taken from the sump of the 1^{st} column represent a diverse mixture of organic compounds. From this mixture compounds may be separated which have a high market value. At the same time, this is a way of feedstock recycling. Also interesting in this context is the CO₂-balance, because for the production of these substances from fossil resources such as crude oil, along with the feedstock base a lot of process energy is consumed, which is saved in this case.

In order to obtain good yields in the separation of said mixtures preferably combinations of rectification, extraction, and sorption must be used.

The proportions that are not separable and/or for which no adequate market exists, can be used for energy, or may be sold to processors, such as refineries. Currently it is required, however, that the mixtures are free from heteroatoms. This can be achieved by hydrogenation, possibly in conjunction with hydrocracking.

In this case, hydrogenation and hydrocracking, respectively, or the sale to refineries is recommended as the downstream process.

The sediment is preferably eluted, i.e., washed. If the sediment is not processed on metals, it is preferred to add sulfide ions to the wash water to convert heavy metal salt that are present into sparingly soluble sulfides to ensure that the substance mixture is suitable for landfill disposal.

In many cases a workup focusing on metals will be worthwhile, because many heavy metals (usually in the form of soaps) are introduced particularly by plastics such as PVC. Usually, they achieve high market prices.

In addition to chemical separation processes, in particular electrochemical separation processes may be used for the separation of the individual metals.

Not at every site it is economically viable, to drive the material yield quantitatively with respect to the organic constituents. The organic components present in the sump of the primary reactor which have been converted into liquid form can also be used for thermal processes.

Preferably, this is done by means of thermal gasification, particularly preferably according to modified black liquor gasification processes wherein the gasification is carried out under pressure so that the condensation of the solution water of can be used after gasification. The gas produced by gasification can be processed further for energy or material purposes.

Depending on specific site conditions, a purely thermal processing, or a maximum substance recovery, or a combination of both may be appropriate.

Hereinafter, for example, the treatment and the possible reaction products of some waste treated according to the invention are described.

### Biopolymers

### Cellulose

In the hot, aqueous, alkaline solution cellulose undergoes a molecule degradation (peeling reaction). This leads to the formation of polyfunctional compounds. Particularly, hydroxyl and carboxyl groups are dominant in the products.

### Lignin

Lignin is degraded substantially into different phenol derivatives. Due to the high acidity of the phenols (resonance stabilization, inductive effects of the aryl residues) the equilibrium of salt formation is shifted far to the side of the salts. Thus, these products are readily soluble in polar solvents.

### Fatty Acids

The biogenic fatty acids saponify to carboxylic acid salts and propanetriol, under the given conditions, the alcohol components are almost quantitatively present in the deprotonated form also. With very long residence times gradual degradation of the carboxylic acids occurs by decarboxylation.

### Proteins

Proteins are degraded into a large number of compounds. This results also in the formation of many smelly compounds, e.g. thiols, amines, ammonia.

### Heteroatom-Free Bulk Plastics/Polymers

### Polyolefins

Polyolefins are hardly altered by solvolysis, they melt, with polypropylene in particular being very viscous at temperatures of about 200° C. It is therefore advisable to add oil as a solvent and to lower the viscosity in favor of pumpability.

### Bitumen Masses

Essentially, Bitumen is only melted and floats as a low-viscosity phase (at the reaction temperature).

### Tar Masses

Tars, which contain mainly aromatics are partly converted into soluble compounds, however, the bulk (at the reaction temperature) is present as low-viscous, floating phase.

### Polystyrene

For PS, a purely thermally induced degradation to monomers or oligomers is common only starting at 250° C. Nevertheless, under certain conditions, there is still degradation due to the impact of organic compounds introduced in the molecular composite and by the mechanical movement of the stirrer.

### Heteroatom-Containing Bulk Plastics

Polyethylene terephthalate/polyester

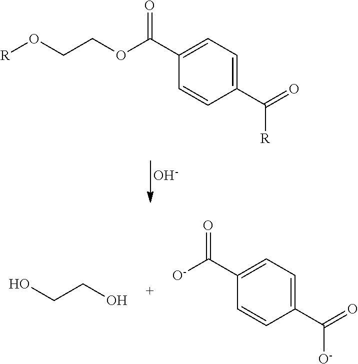

Chemically speaking, PET is an ester of terephthalic acid and ethanediol, wherein under the conditions of the solvolysis it is hydrolyzed to form ethanediol and terephthalic acid. According to the thermodynamic equilibrium the two products are present mainly in their salt form. Terephthalate is readily soluble when heated.

### Polyvinyl Chloride

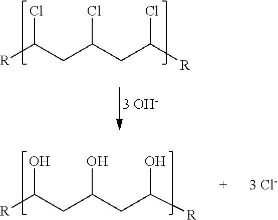

The aim of the solvolysis of PVC is the substitution of the organically bound chlorine. Organically bound chlorine poses high risks when it is processed with heating, on the one hand HCl cleavage may occur by elimination (pronounced above 200° C.), which may lead to new (toxic) compounds by substitution reactions. On the other hand, the covalent C—Cl bond having approximately equal electronegativities is prone to hemolytic, heat-induced bond breakage. The resulting radicals then lead to uncontrollable reactions that may lead to formation of highly toxic products. To prevent this, it is ensured that during the solvolysis the below reaction takes place. The driving force of the reaction is the basicity of the leaving group (Cl- better leaving group due to low basicity of the conjugate base)

### Polyurethane

Unlike, for example, PET, polyurethanes cannot be cleaved to form monomers, rather they are cleaved to form the corresponding alcohol component and diamines. From said diamines, diisocyanates can be prepared by reaction with phosgene.

The following example 1,4-butanediol is formed as the alcohol component:

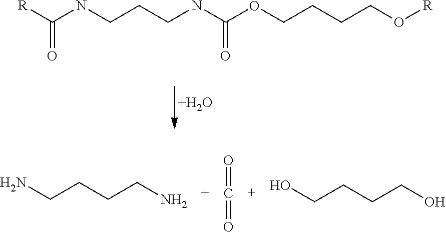

### Polyamide

The hydrolysis of polyamide also leads to diamine and alcohol.

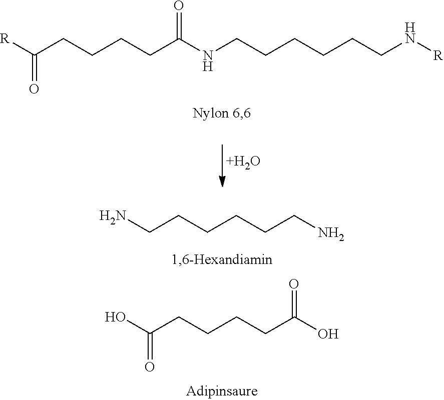

[1,6-Hexandiamin: 1,6 hexanediamine]
[Adipinsäure: adipic acid]

### Fillers Made of Plastics

### Inorganic Fillers

Inorganic fillers are used, e.g. to increase the strength of plastics, for example glass fiber reinforced plastics, to increase the thermal load, or simply for “making go further” of the plastics.

Used are, inter alia
silicic acid, also ensiled,
quartz flour,
silicates and mixed silicates, respectively, of alkali metals and alkaline earth metals, aluminum, iron and zirconium,
Oxides or mixed oxides of alkaline earth metals, aluminum, silicon and zinc
hydroxides and mixed hydroxides, respectively, of calcium, magnesium and aluminum,
carbonates and mixed carbonates, respectively, of calcium, magnesium and aluminum,
sulfate and mixed sulphates, respectively, of calcium and barium,
calciumsulfo aluminate,
aluminum powder and aluminum bronze,
graphite,
glass fibers,
glass microspheres,
dialuminum hexamagnesium carbonate hexadecahydroxide tetrahydrate,
magnesium sodium fluoride silicate,
heavy metals in the form of soaps.

These substances accumulate partly in slightly modified form in the sediment after being dissolved out of the plastic matrix.

In some plastics, the proportion of fillers can exceed 50%.

### Other Accompanying Substances of Plastics

Flame Retardants

The most important representatives are polybrominated diphenyl ether (pentaBDE, octaBDE, decaBDE), TBBPA and HBCD. Until the 70s, in addition, polybrominated biphenyls (PBBs) were used as flame retardants. Chlorinated flame retardants include, for example, chlorinated paraffins and mirex. With the exception of TBBPA these substances are used only as an additive flame retardant. The main application areas are plastics in electrical and electronic appliances, such as TVs, computers, in textiles, e.g. upholstered furniture, mattresses, curtains, blinds, carpets, in the automotive industry, e.g. plastic components and upholstery and in construction, e.g. insulation materials and installation foams. Particularly, in the event of a fire halogenated flame retardants pose big risks. Upon heat exposure they exhibit a flame-retarding effect wherein the halogen radicals formed in the pyrolysis inhibit the reaction with oxygen. However, high concentrations of polybrominated (PBDDd and PBDFs) or polychlorinated dibenzodioxins and dibenzofurans (PCDDs and PCDFs) are also formed. They are also known under the umbrella term “dioxins” for their high toxicity (“Seveso poison”).

TBBPA constitutes a special case of brominated flame retardants. It is mainly used as a reactive flame retardant, i.e. it is chemically integrated into the polymer matrix, e.g. in epoxy resins of printed circuit boards, and is an integral part of the plastic. Further reactive brominated flame retardants include e.g. bromo- and dibromostyrene, and tribromophenol. Being integrated in the polymer, the emissions of these flame retardants are very low, and are usually pose no risks. Nevertheless, the dioxin formation is still not fundamentally less. To a lower extent, however, TBBPA is also used as additive flame retardant.

All the above-mentioned brominated flame retardants underwent a detailed risk assessment under the EU Existing Substances Regulation 793/93 EEC, or are in the final phase of this risk assessment. As a result, pentaBDE and octaBDE have been banned because they accumulate in the environment, are persistent and toxic, however, these substances will be present in the waste for many years. No risk for humans and the environment was found for decaBDE, no risk to the health was found also for TBBPA. Recently, decaBDE has given rise to discussions, as new research has shown, that the substance can be debrominated under the influence of UV radiation and thus recently banned pentaBDE and octaBDE can be formed.

The potential hazards of flame retardants such as polybrominated diphenyl ethers (PBDEs) and polybrominated biphenyls (PBBs) in relation to the formation of PBDDs/Fs have led to a ban by the EU (WEEE, RoHS, ElektroG). An exception is decaBDE, which is expressly excluded from this ban. In solvolysis large proportions of these halogenated organic compounds are modified by similar mechanisms as described for PVC, so that the hazard potential decreased significantly during thermal decomposition reactions.

### Inorganic Accompanying Loads

### Inorganic Flame Retardants

### Organophosphorus-Based Flame Retardants:

In this class of compounds typically aromatic and aliphatic esters of phosphoric acid are used, such as:
TCEP (tris(chloroethyl) phosphate),
TCPP (tris(chloropropyl) phosphate),
TDCPP (tris(dichloroisopropyl) phosphate),
TPP (triphenyl phosphate),
TEHP (tris-(2-ethylhexyl) phosphate),
TCP (tricresyl phosphate),
ITP (isopropylated triphenyl phosphate), mono-, bis- and tris(isopropylphenyl) phosphate with different degrees of isopropylation,
RDP (resorcinol bis(diphenyl phosphate)),
BDP (Bisphenol A bis(diphenyl phosphate)).

These flame retardants are used, for example, in soft and hard PUR foams in upholstered furniture, car seats, or construction materials. More recently, BDP and RDP are increasingly used as substitutes for octaBDE in electrical device plastics.

### Inorganic Flame Retardants are for Example:

aluminum hydroxide (Al(OH)₃), the world's most widely used flame retardant (also called ATH for “aluminum trihydrate”). It exerts a cooling and gas-diluting effect by eliminating water, however, it must be added in high concentrations (up to 60%).
Magnesium hydroxide (Mg(OH)₂, MDH, “magnesium dihydrate”) is a mineral flame retardant with higher thermal stability than ATH, but with the same mode of action.
Ammonium sulfate ((NH₄)₂SO₄) and phosphate ((NH₄)₃PO₄) are diluting the gas in the flame by elimination of ammonia (NH₃), which burns forming water and various nitric oxides and removes the oxygen from the flame. At the same time they cause the formation of a protective layer of the resulting sulfuric acid (H₂SO₄) and phosphoric acid (H₃PO₄), respectively, which can interrupt the radical chain reaction as one of their functions. In addition, the acids are non-flammable, highly hygroscopic and have high boiling points. Therefore, they condense in the cooler region of the flame and are deposited on the material. In addition, phosphoric acid forms meta- and polyphosphoric acid by dehydration, which have even higher boiling points.
Red phosphorus forms a layer of phosphoric and polyphosphoric acids on the surface and causes it to swell (intumescence). This layer acts as insulation and protects the material from oxygen entry. The phosphates formed here have the same characteristics as those derived from ammonium phosphate.
Antimony trioxide (Sb₂O₃) acts only as a synergist in combination with halogenated flame retardants. The disadvantage is its catalytic effect on the dioxin formation in case of fire.
Antimony pentoxide (Sb₂O₅) acts similarly to Sb₂O₃, as a synergist.
Zinc borate (see borates) acts cooling and gas-diluting, inter alia, by water elimination. Zinc compounds can act also synergistically, partially replacing the more hazardous antimony trioxide.
During World War II, slaked lime (Ca(OH)₂) was used as a flame retardant for the wood of the roof truss. It binds initially carbon dioxide from the air with elimination of water forming calcium carbonate (CaCO₃). As a protective coating it makes it difficult for oxygen to enter.

The inorganic flame retardants are released partly in somewhat altered chemical form (e.g. as calcium phosphate) after dissolution of the polymer matrix and settle (sediment).

### Metals

Metals in oxidation state 0 (possibly with an oxidized surface) are mainly from components of technical devices, e.g. in the form of wire, housing parts, screws, metal foils, etc.

Metals which act largely precious with respect to the solution, such as iron, copper, nickel, etc. sediment. Before, they are freed of any composites, cable insulation, from cast materials, etc., by converting the surrounding plastic, or the surrounding biopolymer (nails in wood), etc. into a liquid form.

Metals which act non-precious with respect to the solution are chemically modified.

Aluminum forms an aluminate with evolution of hydrogen. After aging, they form sedimenting compounds, e.g. oxyhydrates.

Zinc, tin, arsenic, etc. initially form soluble compounds, from which then the corresponding sulfides can be precipitated.

The following figures illustrate the subject of the invention without limiting it to the specific embodiments shown herein.

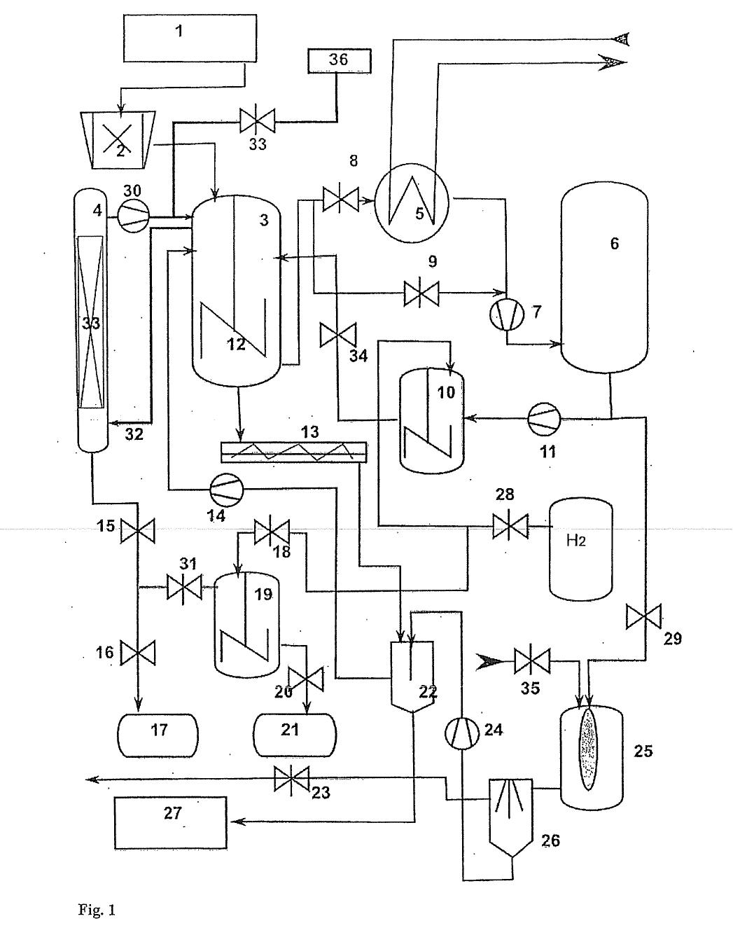

FIG. 1 shows a schematic representation of a plant for carrying out the process according to the invention;

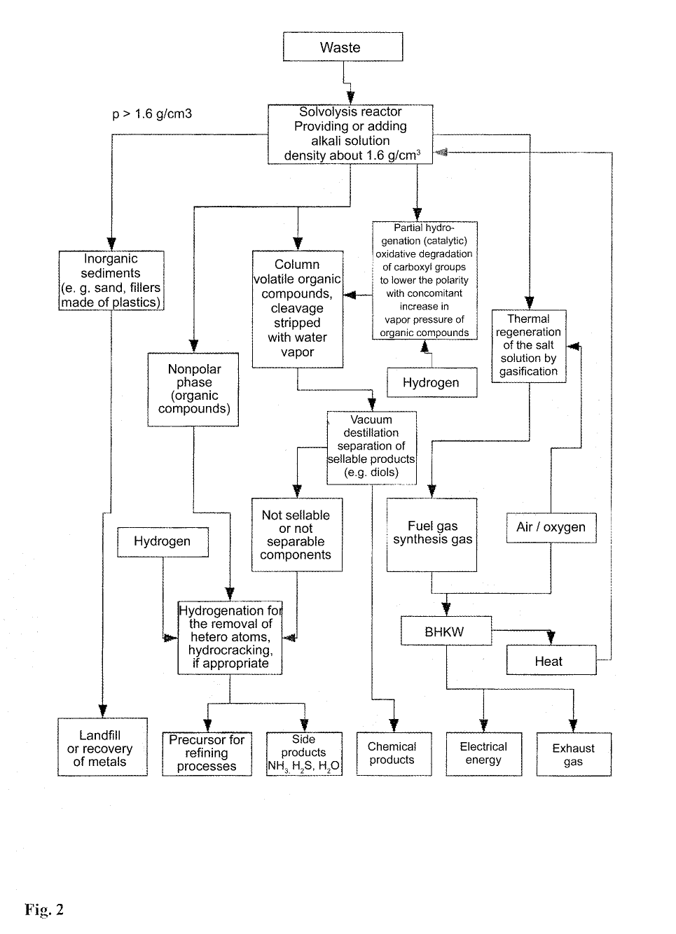

FIG. 2: Flow diagram of the process according to the invention;

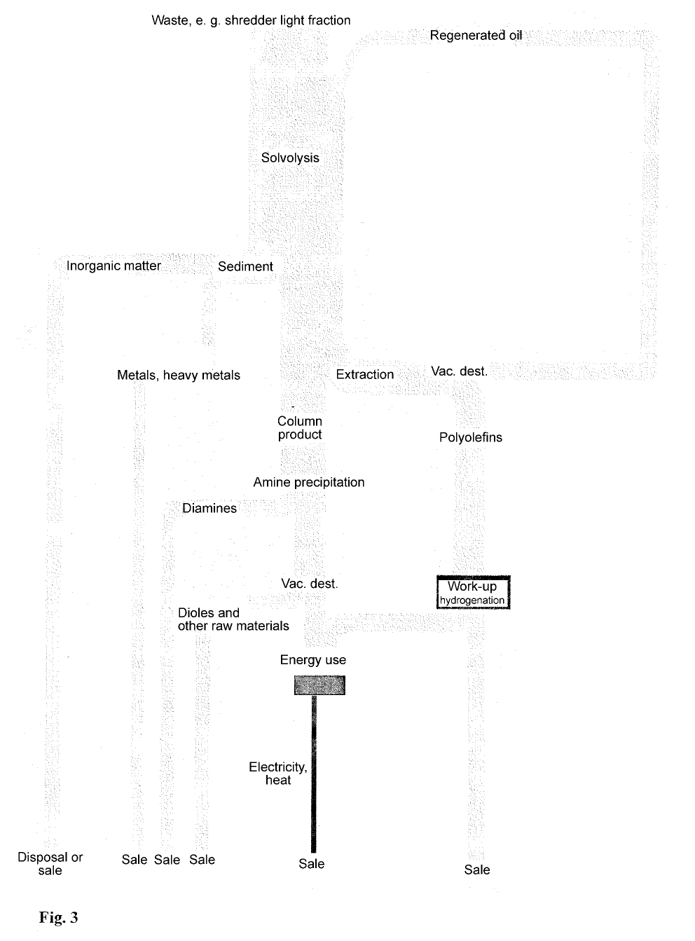

FIG. 3 shows a Sankey diagram for the process according to the invention;

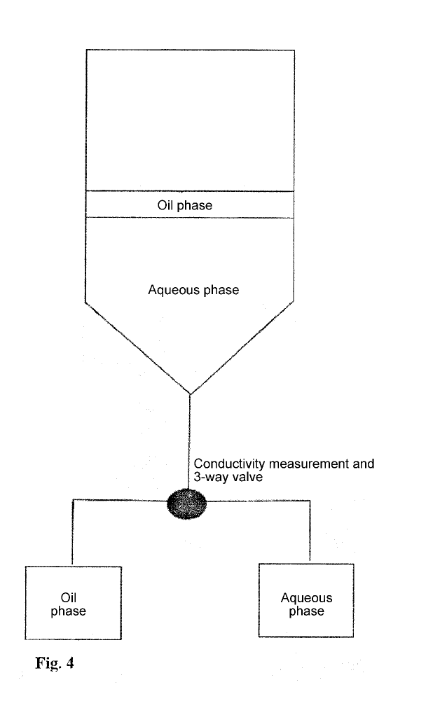

FIG. 4 shows a process diagram for the separation of the aqueous and the organic phase from the reactor;

FIG. 5 shows a diagram of pressure, temperature and solvent concentration.

Thus, FIG. 1 shows a schematic representation of a plant for carrying out the process according to the invention, wherein the material to be processed is added from the transport vessel 1 (optionally after intermediate storage) into reactor 3 after an optional post-shredding 2.

Here, the feeding can take place quasi-continuously (option 1->semi batch) (e.g. by a compacting screw or piston press), wherein the digestion solution is provided, or the vessel lid of the reactor is opened (option 2->batch mode).

In the latter approach, the digestion solution had to be pumped previously into the reservoir 6, the valves 8, 9 and 34 must be closed in this case. Before opening the lid, the vessel must be depressurized to displace odors, toxic gases and flammable vapors, if present, from the container by steam or ventilation.

In the case of batch operation, the alkaline solution is added again after filling and inertization, if appropriate.

The reactor is equipped with a mechanical stirrer, preferably with an anchor stirrer. It is also possible to run the reactor as a circulation reactor, however in this case the entry of the solution in the container (preferably tangentially) must ensure a sufficient movement of the reactants. In the above-mentioned scheme, the reactor is designed as stirrer tank reactor and as a circulating reactor. Supplying heat to the process is preferably carried out by a heat exchanger 5 within the circulation which leads from container 3 via container 6->10->back into container 3.

Optionally, the reactor can also be designed as a double jacketed vessel and heated by steam, thermo oil or other appropriate forms of heat supply.

After the decomposition or melting of the organic components, the inorganic substances that are tight fit connected (e.g., insulated wires) or bound in an organic matrix (e.g., plastic compounds) are dissolved and sediment due to their higher density than the digestion solution.

These sediments are removed continuously via a discharge device 13 or in batch mode after opening of the reactor. After the discharge, the sediments with organic matter-loaded digestion solution adhering thereto, are washed using freshly regenerated digestion solution 22. Subsequently, the sediment is washed with water and disposed of in a landfill or worked up for metal recovery (e.g., electro-refining).

Column 4 assumes a key role in the process. Its task is to separate from the dragging steam the proportion of organic components contained in the water vapor, whose vapor pressure is lower than that of water. This requires forced circulation. The column must be designed as a packed column 33.

The dragging steam depleted of organics is always fed back into the reactor 3, so that the dragging steam can be re-loaded with organic matter.

The gases and vapors having higher vapor pressures than water contained in the dragging steam would lead to a continuous pressure increase, therefore they are led into a condensation apparatus 31 via a pressure regulating valve. Inevitably some water vapor is discharged in the course.

Unless the system already has an excess of water anyway due to the moisture of the starting material, or by cleavage reactions with elimination of water, the condensed water should be fed back into the reactor. The discharged gases must be freed from interfering gases and can then be used as fuel gases, or simply flared.

The organic liquids collected in the sump of column 4 can be further separated by distillation. Preferably, this distillation is carried out in vacuo, partly because the spreading of the vapor pressure difference is larger, and on the other hand, many products have very high boiling points, so that the necessary distillation temperatures would lead to decomposition.

For the fractions, which cannot be separated at a purity sufficient to achieve product qualities, or for which there is no sufficient market, a hydrogenation 19 must be provided. Preferably, this hydrogenation is carried out catalytically. By doing so, products are produced which are largely free of heteroatoms such as O, S, and N. These products are then accessible, e.g. for typical refinery processes and have a market value.

Measures for increasing the yield of the column product by reaction in the container 10 are described in more detail in the respective section.

High-polymeric organic substances, which are present in reactor 3 as a floating phase, after being pumped into container 6 can be removed therefrom. Preferably by pumping (control via the conductivity of the liquid). These substances contain hardly any heteroatoms and can also be processed further in refineries.

FIG. 3 shows a schematic process diagram for separation of the aqueous and the organic phase from the reactor. Here, in the case of a batch circulating reactor, separation can be performed continuously via a gravity separator (oil separator), a hydrocyclone or similar technical facilities.

The depletion of organic matter based on the separation by vapor pressure or extraction has its limits. Particularly polar substances prone to salt formation (particularly carboxylates) are not suitable for these two options.

For this reason, the solution is enriched with these substances, which would lower the pH value and ultimately would lead to a standstill of many solvolysis reactions.

Therefore, the organic components must be removed from the solution, and thus the solution must be regenerated again.

There are three approaches, which can be performed individually or in combination.

Kolbe Electrolysis:

The polarity can be decreased dramatically by removal of the functionality by a one-electron oxidation of a carboxyl group R—COO— to R—COO. radicals, which degrade by decarboxylation to CO₂ and R. and which in turn react with R′. to form the compound R—R′. The resulting substances are now amenable to distillation and extraction, respectively, and thus may be separated from the mother liquor

Hydrogenation:

Using a catalyst (preferably Raney nickel) polar substances can be hydrogenated and partially hydrogenated, respectively, to make them amenable to extraction and distillation, respectively.

Thermal Processes

The solution can also be treated using thermo-chemical methods such as pyrolysis, combustion, gasification, refining to recover the alkalis. After combustion processes the carbonate (preferably potassium carbonate) is present again and is re-used as educt.

FIG. 5 shows a diagram for pressure, temperature and solvent concentration. For control reasons it has proven successful to control the temperature and the pressure to obtain the corresponding concentration. I.e. excess water is leaving the system. Only when the water-consuming processes (e.g. hydrolyses) predominate, an addition of water is necessary. By comparison with the p/T curve, this may be determined by ICA.

In addition to impacting the equilibrium pressure in the system, the reaction temperature mainly impacts the following parameters:
the kinetics of the solvolysis processes—particularly slow reactions are dehalogenation (cf. PVC) and the degradation of polysaccharides =>required reaction time and therefore space/time yield
the secondary decomposition reactions (e.g. in diols)=>yield reduction,
the viscosity of the feedstocks, at high temperatures the educts are less viscous, more moveable thus finer distribution (emulsion)=>space/time yield
the partial pressure of the organic components in the dragging steam =>column efficiency/reflux ratio

Preferably, the reaction temperature is 180 to 220° C. The reaction times are then in the range of about 10 to 20 hours (batch mode) and the equilibrium pressures are at 4 to 10 bar.

### REFERENCE SIGN LIST

1—Reservoir (usually container)
2—Crushing device (optional)
3—Solvolysis reactor
4—Packed column (preferably Raschig rings)
5—Heat exchanger (with option circulation reactor), alternative: direct heating solvolysis reactor
6—Reservoir solution (several reactors can be connected)
7—Pump for solution
8—Check/control valve circulation solution (if the solution is not intended to flow through the heat exchanger (control function))
9—Check/control valve for bypass for heat exchanger
10—Reactor for partial hydrogenation (may be combined with Kolbe electrolysis, if appropriate [when using nickel electrodes and hydrogen as a redox partner ->nascent hydrogen at the Ni surface])
11—Pump for solution, feed for the hydrogenation reactor
12—Stirrer solvolysis reactor
13—(Continuous) sediment discharge device
14—Pump for thermally regenerated solution
15—Check/control valve column sump
16—Check/control valve product removal
17—Vessel for column product
18—Check/control valve hydrogen hydrogenation reactor for hydrogenation column product
19—Hydrogenation reactor for column product
20—Check/control valve hydrogenation reactor, product removal
21—Container for hydrogenated column product
22—Gravity separator for sediment after gasification (e.g. fully annealed aluminum oxide, which was in solution as aluminate prior to gasification) and pre-cleaning of the d=sediment from the solvolysis reactor (post-washing with water for the removal of alkali usually required)
23—Check/control valve synthesis gas (in gasification) and exhaust gas (in combustion)
24—Pump thermally regenerated solution to gravity separator
25—Carburetor or combustion device
26—Quencher to separate salt mist/gas and cooling to equilibrium pressure temperature.
27—Vessel for sediment from 22
28—Check/control valve for hydrogen
29—Check/control valve for solution for thermal regeneration
30—Steam ventilation (if possible axially due to beating of water by saturated steam)
31—Check/control valve for hydrogenation vessel column product
32—Steam line to the column (dragging steam containing organic matter)
33—Control valve system pressure (if degradation gases arise they must be discharged from the system)
34—Feedback by Kolbe electrolysis/(partial) hydrogenation regenerated solution
35—Gasification/combustion air or oxygen supply control.
36—Condenser for steam entrained in gases during discharge, attached thereto (not shown) condensate feedback to solvolysis reactor (if necessary, otherwise excess water can be discharged from the system here, and exhaust treatment (binding amines, e.g. ethylamine, ammonia, etc.)

I claim:

1. A process for the treatment of waste in a reactor (3) comprising the following steps:
Combining the waste to be treated and an aqueous, alkaline solution to form a reaction mixture;
Heating the reaction mixture to a temperature range between 140° C. to 250° C. and a pressure between 3 bar and 12 bar to produce at least one aqueous, organic phase, a gas phase and optionally a solid phase;
Removal of the gas phase from the reactor (3);
Separation of the organic and aqueous components of the gas phase, in particular by a rectification column (4);
feeding the aqueous components of the gas phase back into the reactor (3).

2. A process according to claim 1, characterized by the separation of the solid phase from the reaction mixture.

3. A process according to claim 2, characterized by washing the solid phase after separation.

4. A process according to claim 1, characterized in that the waste comprises at least one of the following components: solid and liquid biomasses or products produced from them, plastics (polymers), inorganic materials such as minerals, metals and compounds thereof, solid or liquid pollutants and/or hazardous materials, such as heavy metals and compounds thereof, halogen-substituted organic compounds, such as flame retardants, cellulose-containing raw materials and waste, fatty acid, biopolymers, bitumen masses, tar masses, and combinations thereof and the like.

5. A process according to claim 1, characterized in that the alkaline solution contains carbonates, particularly potassium carbonate.

6. A process according to claim 5, characterized in that said solution is a saturated potassium carbonate solution and/or the density of the solution is 1.5 to 1.6 g/cm³.

7. A process according to claim 1, characterized in that a water-insoluble, organic auxiliary (extractant) is added to the reaction mixture.

8. A process according to claim 7, characterized in that the organic auxiliaries are lubricating oils, motor oils, aliphatic hydrocarbons or the like.

9. A process according to claim 7, characterized by the separation of the aqueous, alkaline phase and organic phase by extraction and/or decantation.

10. A process according to claim 7, characterized by the treatment of the aqueous alkaline phase and/or the organic phase hydrogenation and/or Kolbe synthesis.

11. A process according to claim 7, characterized by vacuum distillation of the organic and/or aqueous phase.

12. A process according to claim 1, characterized in that the alkaline solution is provided in the reactor (3) and a quasi-continuous feeding of the waste takes place in semi-batch operation, in particular by means of compacting screws and piston presses.

13. A process according to claim 1, characterized in that the reactor (3) is a stirrer tank having a mechanical stirrer (12) and/or a circulation reactor.

14. A process according to claim 1, characterized by maintaining the pressure and the temperature for a period of 0.5 h to 30 h, preferably from 2 h to 20 h and in particular for more than 10 h.

15. A process according to claim 1, characterized by crushing the waste to be treated to a given particle size which, in particular, has at least one edge length of 0.1 to 10 cm.

16. A process according to claim 1, characterized in that the feeding of the waste takes place in batch mode by opening the reactor (3) and inputting the waste, and subsequently adding the alkaline solution.

17. A process according to claim 1, characterized in that the supply of heat during the process takes place by at least one heat exchanger (5) or by a heat transfer medium used in the process, particularly steam or thermal oil.

18. A process according to claim 1, characterized in that the inorganic components are removed continuously or batchwise by at least one discharge device and are washed subsequently by processed, alkaline solution and/or water.

19. A process according to claim 1, characterized in that metals are recovered from the inorganic components by means of electric refining and/or galvanic and/or chemical and/or mechanical processes.

20. A process according to claim 1, characterized in that the rectification column (4) is a packed column.

21. A process according to claim 1, characterized in that the pressure after the rectification column (4) is held constant using a pressure regulator.

22. A process according to claim 1, characterized in that the organic components are separated from the dragging steam through transfer into a condensing apparatus, and the dragging steam depleted of organic constituents is fed back into the process.

23. A process according to claim 1, characterized in that a partial hydrogenation, in particular using Raney nickel as catalyst, of at least a portion of the organic components occurs at too low a vapor pressure, is lowered by the polarity of said organic components and thus increases the vapor pressure thereof.

24. A process according to claim 1, characterized in that the organic components present in liquid form are subjected to hydrogenation, in particular using Raney nickel as catalyst, for the removal of heteroatoms and/or are subjected to hydrocracking.

25. A plant for carrying out one of the process for the treatment of waste of claim 1 with a reactor (3), means for supplying wastes, means for supplying an alkaline solution, optionally means for supplying an extractant, means (32) for removing of a gas phase from the reactor (3) and means (4) for separating the organic and/or aqueous components from the gas phase and means for feeding the aqueous components back into the reactor (3).

26. A plant according to claim 25 further comprising means for removal of the aqueous solution into a reservoir (6), a reactor for the partial hydrogenation (10).

27. A plant according to claim 25 further comprising a discharge device (13) for the sediment from the reactor (3).
